(19) United States
(12) Patent Application Publication
(10) Pub. No.: US 2021/0259914 A1
(43) Pub. Date: Aug. 26, 2021

(54) SYSTEMS AND METHODS FOR PAIN MANAGEMENT

(71) Applicant: Omega Dolor Inc., New York, NY (US)

(72) Inventor: Tabitha Holbert, New York, NY (US)

(73) Assignee: Omega Dolor Inc., New York, NY (US)

(21) Appl. No.: 17/180,710

(22) Filed: Feb. 19, 2021

Related U.S. Application Data
(60) Provisional application No. 62/980,089, filed on Feb. 21, 2020.

(51) Int. Cl. A61H 23/02; A61F 13/02

(52) U.S. Cl. CPC A61H 23/02; A61F 13/0233; A61H 2201/0119; A61H 2201/0107; A61F 2013/0028; A61H 2201/5007; A61H 2201/1685; A61H 2201/169; A61H 2201/1654; A61H 2201/1207

(57) ABSTRACT

Systems and methods for pain management are disclosed herein. In some embodiments, a vibrating pain management device includes a vibration generator attachable to a wearable patch having first and second arms for attaching the patch to a wearer. The patch is at least partially stretchable.

### RELATED APPLICATIONS

This application claims the benefit under 35 U.S.C. § 119(e) to U.S. Provisional Application Ser. No. 62/980,089, filed Feb. 21, 2020 and entitled “SYSTEMS AND METHODS FOR PAIN MANAGEMENT”, which is herein incorporated by reference in its entirety.

### FIELD

The disclosed embodiments relate to pain management.

### SUMMARY

According to one embodiment, a pain management device includes a patch having a body with a first side arranged to contact a body of a wearer, a second side opposite the first side, and first and second arms arranged to attach the patch to the body of the wearer, wherein at least a portion of each of the first and second arms is stretchable, and a vibration generator arranged to be attached to the body of the patch.

According to another embodiment, a kit includes a patch having a body with a first side arranged to contact a body of a wearer, a second side opposite the first side, and first and second arms arranged to attach the patch to the body of the wearer, and a first vibration generator arranged to be removably attachable to the body of the patch.

According to another embodiment, a method includes attaching a vibrating pain management device to a body of a wearer, the pain management device including a patch and a vibration generator attachable to the patch, at least a portion of the patch being stretchable, and operating the vibration generator to provide a vibration to the body of the wearer.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a top view of a vibrating pain management device according to embodiments disclosed herein;

FIG. 2 is a cross-sectional side view of the device of FIG. 1;

FIG. 3 is a top view of a patch of the device of FIG. 1;

FIG. 4 is a cross-sectional side view of the patch of FIG. 3;

FIG. 5 is an enlarged schematic side view of a portion of a patch according to some embodiments;

FIG. 6 is a cross-sectional side view of a vibration generator according to some embodiments;

FIG. 7 is a bottom view of a vibration generator according to some embodiments;

FIG. 8 is a top view of the vibration generator of FIG. 7;

FIG. 9 is a top view of a patch according to some embodiments;

FIG. 10 is a perspective view of a vibration generator according to some embodiments;

FIG. 11 is a cross sectional side view of the vibration generator of FIG. 10;

FIG. 12 is a perspective view of the vibration generator of FIG. 10, with a housing of the vibration generator show in transparency;

FIGS. 13A and 13B illustrate a mobile device application usable with a vibrating pain management device according to some embodiments; and

FIG. 14 is a schematic view of a computer system according to one embodiment.

### DETAILED DESCRIPTION

Women make up more than half of the population, and many of those women suffer from menstrual cramps. For example, many women experience some type of abdominal pain or cramping every 21 days. For some women, symptoms may last only one day, or even only part of a day, while for other women, the symptoms may last multiple days. For some women, the pain may be simply an inconvenience. However, for other women, the pain may be so severe that it may be disruptive to their daily lives. Traditionally, relief for menstrual cramps includes pain relievers, birth control pills, herbal remedies, and/or heat compresses.

The inventor has recognized that advantages may be realized by providing a wearable vibrating pain management device (also referred to herein as the pain management device, the vibrating patch, the vibrating device, or simply the device) to alleviate pain associated with menstruation. For example, in some embodiments, a user (also referred to herein as the wearer) may attach (e.g., mount) a vibration generator (also referred to herein as a vibrator) to a patch that may be worn by the user, such as on the abdomen of the user. In some embodiments, the vibration generator may include a vibrating disc that the user may mount to the wearable patch. As described herein, the vibration generator may have other suitable shapes in other embodiments.

The inventor has also recognized that advantages may be realized by providing a vibrating pain management device with a vibration generator that the user may modulate. For example, the inventor has recognized that advantages may be realized by leveraging knowledge and understanding of Gate Theory to target menstrual pain. Without wishing to be bound by theory, under Gate Theory, non-painful input close to the nerve “gate(s)” to a painful impact may prevent painful sensation from travelling, such as to the central nervous system. In some embodiments of the present disclosure, the user may increase or decrease the intensity of the vibrations of the vibration generator to address different levels of pain. In some embodiments, the vibration generator may be arranged to stay at the same, set level of intensity for a period of time. In other embodiments, the vibration generator may have different vibration sequences that a user may select to address pain. For example, in some embodiments, the vibration sequence may include a wave of vibrations. In still other embodiments, the vibration generator may produce randomized vibrations to further enhance the benefits provided from Gate Theory.

The inventor has further recognized that advantages may be realized by allowing the user to catalog pain information (e.g., in real-time), such as via an application on a mobile device. In some embodiments, the real-time pain data may be stored in the mobile device and/or on a remote computer, along with vibration information of the pain management device. In some embodiments, the user may relay the collected pain data to a physician to assist with creating a treatment plan, if necessary. In some embodiments, the pain data may be used by the mobile application to recommend a vibration intensity and/or sequence based on the past use of the pain management device. The user may thereafter adjust the intensity and/or sequence of the vibration generator based on the recommendation. The pain data also may be usable by the mobile application to automatically control the vibration generator based on a specified pain level and past use of the vibration patch. For example, in some embodiments, the vibration generator may be controllable via the mobile application, with the mobile application automatically adjusting the intensity and/or sequence of the vibration in response to a specified pain level of the user.

In some embodiments, the vibration generator may include an integrated real time clock. In some embodiment, the vibration generator also may include a memory module arranged to record timestamped usage of the vibration generator. In some embodiments, the memory module may record the timestamped usage locally. In some embodiments, the timestamped usage may be transmitted from the vibration generator at a later time, such as to a mobile application.

In view of the above, embodiments disclosed herein include a vibrating pain management device that a user may wear to alleviate pain associated with menstrual cramps. As will be appreciated, while embodiments have been shown and described for relieving pain associated with menstrual cramps, the disclosed arrangements may be used for relieving other types of pain, for both men and women. For example, the method and systems may be used to relieve back, neck, and/or shoulder pain in other embodiments.

In some embodiments, the vibrating pain management device may include a vibration generator that a user may attach to a patch that is wearable by the user. In some embodiments, the patch, or at least a portion of the patch, may be disposable, with the user removably attaching a vibration generator to the patch. The patch also may be reusable in some embodiments. In some embodiments, the vibration generator may be reusable, although the vibration generator also may be disposable (or at least partially disposable). The vibration generator also may be permanently attached to the patch, with the vibration generator and patch both being reusable in some embodiments.

In some embodiments, the vibration generator may include a vibrating disc, although the vibrating source may have other suitable shapes, such as a triangle, square, other polygonal, or other suitable shape. In some embodiments, more than one vibration generator may be attachable to patch.

In some embodiments, the patch is arranged to be stretchable. For example, in some embodiments, the patch may be formed of a stretchable material such as silicone. In some embodiments only portions of the patch may be formed of a stretchable material. For example, the patch may include one or more flexible regions to allow the patch to be stretched and attached to the user.

In some embodiments, the patch is arranged to be adhered to the skin of a user. In some embodiments, the patch may be formed of a material that is sticky. For example, the patch may be formed of a silicone material with microsuction. In some embodiments, the patch is arranged so that only the body-contacting side of the patch is sticky. In some embodiments, only a portion of the body-contacting side of the patch may be sticky (e.g., only the body contacting side of one or more arms of the patch).

In other embodiments, the patch may include one or more adhesive layers that the user may use to attach the patch to their body. As will be appreciated, after the patch is removed from the wearer, a first adhesive layer may be removed from the patch. During a subsequent use, a second adhesive layer may be used to attach the patch to the wearer.

In some embodiments, the adhesive layer may be formed of a material that is medical grade. In some embodiments, the adhesive layer may be formed of a breathable material. The adhesive also may include a body-safe adhesive. In some embodiments, the adhesive layer may be formed of a waterproof material.

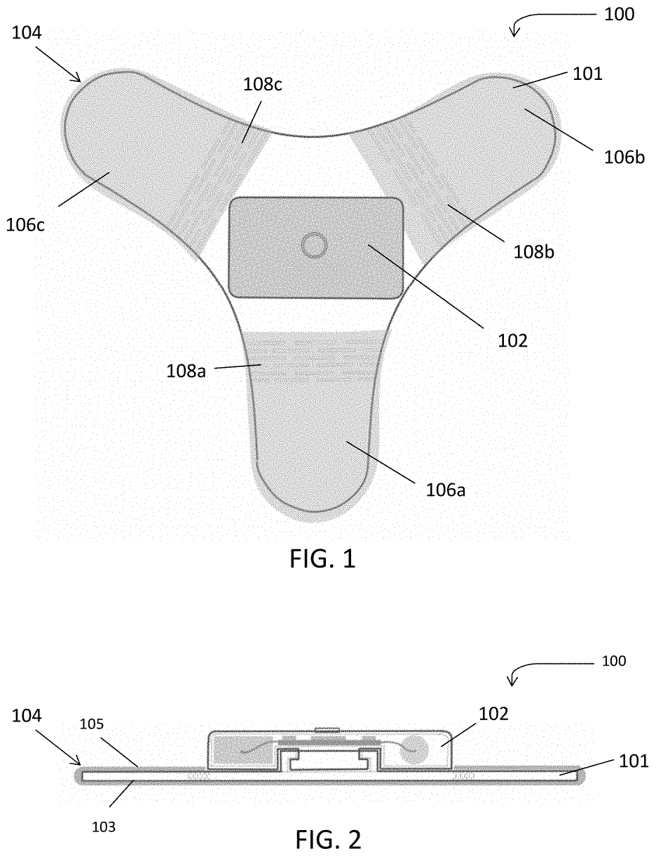

Turning now to the figures, FIGS. 1-4 shows a vibrating pain management device according to embodiments of the present disclosure. As shown in at least FIG. 1, the device 100 may include a vibration generator 102 that is attachable to a patch 104 that is wearable by a user.

In some embodiments, the patch is arranged to be disposable, while in other embodiments, the patch may be reusable.

In some embodiments, as shown in FIGS. 2 and 4, the patch includes a body 101 having a first side 103 arranged to contact a wearer. The patch body also may include a second side 105 to which the vibration generator is attachable. The vibration device also may be attached to other portions of the patch, such as in a pocket formed in between the first and second sides of the body.

In some embodiments, as shown in FIG. 1, a single vibration generator may be attachable to the patch (e.g., to the second side of the patch). In other embodiments, the patch may be arranged to receive more than one vibration generator. For example, in some embodiments, the patch may include a single mount, with the user being able to attach different vibration generators to the same mount. In some embodiments, the different vibration generators may each be arranged to provide different types or levels of vibrations to the user. In another example, the patch may include more than one mount, with the user being able to mount multiple vibration generators to the same patch. For example, a user may attach two, three, or more vibration generators to the patch in other embodiments.

In embodiments in which more than one vibration generator is attachable to the patch, the vibration generators may be the same shape and size, although the vibration generators may vary in shape and size from vibration generator to vibration generator. For example, in an illustrative embodiment, a vibrating disc may be attached to the same patch as a vibrating square, such as to the same mount.

In some embodiments, the different vibration generators may include different modules (e.g., power modules or sequence modules) such that the user can customize and/or enhance vibrations. In embodiments in which more than one vibration generator is attached to the patch at the same time, each of the vibration generators may be individually modulated. For example, the intensity and/or vibration pattern of the first vibration generator (e.g., the vibrating disc) may be selected to be different than that of the second vibration generator (e.g., the vibrating square).

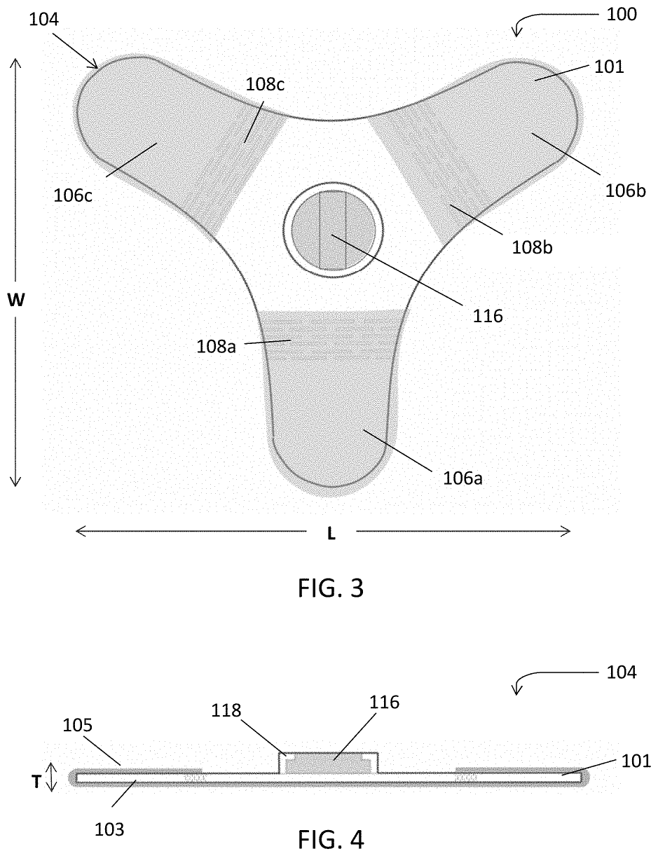

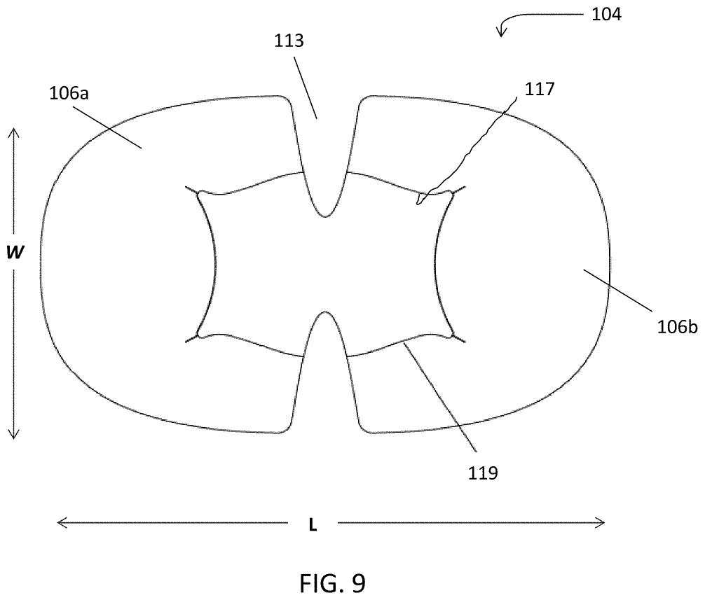

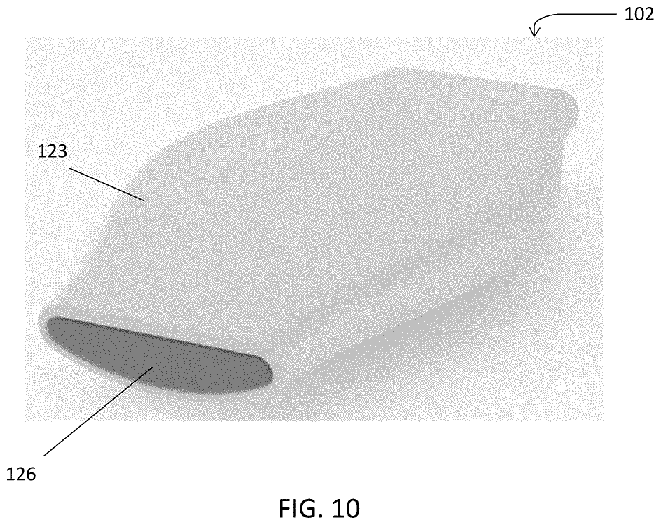

In some embodiment, as shown in FIGS. 1 and 3, the patch may be substantially triangular in shape. The patch also may have other suitable shapes in other embodiments. For example, as shown in FIG. 9, the patch 104 may be substantially elliptical in shape. The patch also may be circular, oval, square, other polygonal, or other suitable shapes in other embodiments.

In some embodiments, the patch may include a length L (see, e.g., FIG. 3) of between about between about 3 inches and about 4.5 inches. In such embodiments, a width W of the patch may be the same as the length of the patch (see FIG. 3), such as between about 3 inches and about 4.5 inches. In other embodiments (see, e.g., FIG. 9), the length L of the patch may be between about 4.0 inches and about 5.5 inches. In such embodiments, the width W of the patch may be between about 2.0 inches and about 3.5 inches. The patch also may have other suitable lengths and widths in other embodiments.

In some embodiments, the patch may include one or more attachment arms for attaching the patch to the wearer. As shown in FIGS. 1 and 3, for example, the patch may include first, second, and third arms 106a-106c arranged to attach the patch to the wearer. In other embodiments, as shown in FIG. 9, the patch may include only two arms 106a, 106b for attaching the patch to the wearer. Although the patch is shown with two and three arms, the patch may more or fewer arms in other embodiments. For example, in some embodiments, the patch may have only a single arm. The patch also may have four or more arms in other embodiments.

In some embodiments, the number of arms may vary depending upon the overall shape of the patch. For example, in embodiments in which the patch is triangular in shape (see FIG. 3), the patch may include three arms. In embodiments in which the patch is elliptical (or oval) in shape, the patch may include two arms (see, e.g., FIG. 9). In embodiments in which the patch is square in shape, the patch may include four arms.

In some embodiments, the shape and size of the arms may be the same. For example, as shown in FIG. 9, each of the arms may be the same shape and size. In other embodiments, the shape and size of the arms may vary from arm to arm. For example, in some embodiments, the patch may include two smaller arms and a third, larger arm.

In some embodiments, as shown in FIG. 1, a distal end of each arm may be substantially semi-hemispherical in shape. In such embodiments, the distal end of each arm may be curved. The distal ends of the arms also may have other suitable shapes in other embodiments. For example, the distal ends may be square or triangular in shape in other embodiments. As will be appreciated, the distal end of each arm may be the same in some embodiments, although the distal ends may vary from arm to arm.

In some embodiments, as shown in FIGS. 1 and 9, the patch may curve inwardly between adjacent arms. For example, the patch may be concave between the first and second arms. In some embodiments, the patch may include one or more cutouts in between adjacent arms. For example, in some embodiments, as shown in FIG. 9, the patch may have a cutout 113 on either side of the first and second arms. In some embodiments, the shape and size of each cutout may be the same. In other embodiments, the shape and size of each cutout may vary from cutout to cutout. In some embodiments, as shown in FIG. 9, the width of each cutout may be between about 25% and about 40% of the width of the patch.

In other embodiments, the patch may curve outwardly between adjacent arms. The patch also may have other suitable shapes and geometries between adjacent arms in other embodiments.

In some embodiments, such as that shown in FIGS. 1 and 3, the arms may be integrally formed with the rest of the patch. For example, in some embodiments, the patch may include a monolithic structure. In some embodiments, the patch may be formed of a PET material. The patch also may be formed of other suitable plastic materials in other embodiments. In some embodiments, the patch includes a thickness T of between about 0.75 mm and 1.5 mm, such as about 1.0 mm (see FIG. 4).

In other embodiments, one or more of the arms may be removably attachable to the remainder the patch. For example, in some embodiments, one or more arms may be removably attachable to a central portion of the patch or to a mount to which the vibration generator is attached.

In some embodiments, the patch is arranged to be flexible so that the patch may be comfortable worn by the user. In some embodiments, the patch is also arranged to be stretchable. For example, the user may stretch one or more of the arms to secure the patch to their person. In some embodiments, the entire patch may be formed of a stretchable material. In other embodiments, only a portion of the patch is arranged to be stretchable. For example, in some embodiments, only the arms of the patch are arranged to be stretchable. In some embodiments, only some of the arms (e.g., two of the three arms) may stretch.

In some embodiments, such as that shown in FIGS. 1 and 3, the patch may have stretching zones 108a-108b that are arranged to be stretchable. In some embodiments, the stretching zones are arranged to allow the arms of the patch to move such that the patch may be secured to the wearer. For example, the stretching zones may allow a length of one or more of the arms to be increased so that the patch may be comfortably attached to the wearer. As will be appreciated, in such embodiments, the rest of the patch may be formed of a material that is not stretchable, or that is less stretchable than the material of the stretching zones.

In some embodiments, the stretching zones are located at or near a proximal end each arm. In some embodiments, the proximal end of the arm is located at or near a central region of the patch 110, such as where the vibration generator is mounted to the patch. In some embodiments, the arms extend outwardly from the central region of the patch. In some embodiments, each arm may include more than one stretching zone. For example, the arm may include one or more stretching zones positioned along a length of each arm.

Although each arm is shown as having a stretching zone in FIGS. 1 and 3, in some embodiments, only a subset of the arms may include stretching zones. For example, in some embodiments, only one arm may include a stretching zone. In another example, two arms may include stretching zones. As will be appreciated, the number and position of the stretching zones need not be the same for each arm. For example, a first arm may include a single stretching zone at a proximal end of the arm, while a second arm may include two stretching zones positioned along the length of the arm.

In some embodiments, the stretching zones may extend along a width of one or more arms. The stretching zones also may extend along only a portion of the width of the one or more arms. The length and width of the stretching zones may vary from arm to arm in some embodiments, although they may be the same in other embodiments. The shape of the stretching zones also may vary from arm to arm in some embodiments, although they may be the same in other embodiments.

In some embodiments, other regions of the patch may include one or more stretching zones. For example, in some embodiments, the central region of the patch may include a stretching zone in some embodiments.

In some embodiments, the stretching zones may be formed by varying the thickness of the patch. For example, the thickness of the patch in the stretching zone may be less than the thickness of the patch in a non-stretching zone. In other embodiments, the stretching zones may be formed by creating patterns in the patch (see e.g., FIGS. 1 and 3). Stretching zones also may be formed by varying the material of the patch in the stretching zones.

In some embodiments, the patch is arranged to be attachable to a wearer. For example, in some embodiments, at least a portion of the body-contacting side 103 of the patch may be formed of a sticky material. For example, in some embodiments, only the body-contacting side of the arms may be formed of a sticky material. In other embodiments, the entire patch may be formed of a sticky material.

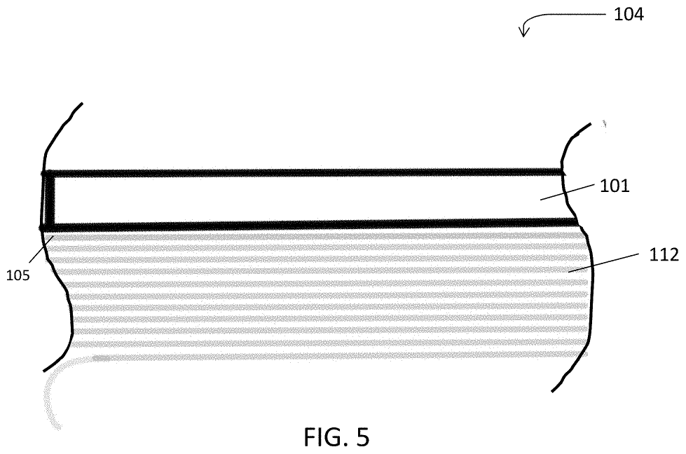

In some embodiments, as shown in FIG. 5, the patch may include a plurality of adhesive layers 112 to attach the patch to the wearer. In some embodiments, the adhesive layers may be attachable to the patch body. For example, in some embodiments, a stack of adhesive layers may be attached to the body-contacting side of the patch. In some embodiments, each of the adhesive layers may be peelable from the wearer after a period of time, such as after the stickiness of the layer has reduced. The adhesive layer also may be removable after a desired period of use of the vibration generator. In some embodiments, each layer may be usable up to about 8 hours. In some embodiments, each layer may be reusable.

The vibration generator is arranged to be mounted to the patch (e.g., on the second side of the patch). In some embodiments, the vibration generator may be removably mounted to the patch. In such embodiments, the vibration generator may include one or more fasteners that engage with corresponding fasteners on the patch.

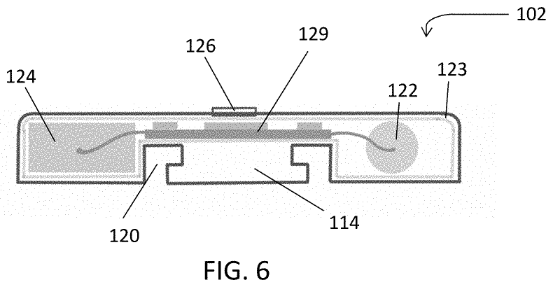

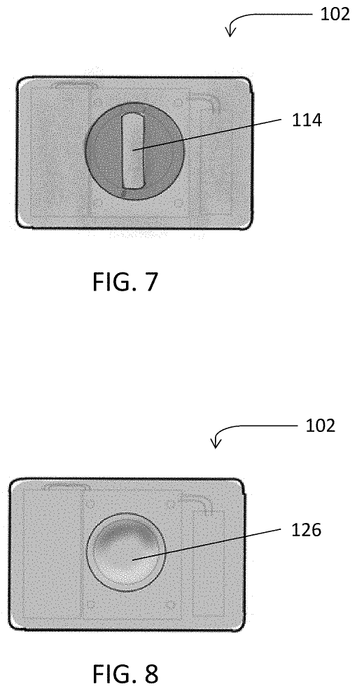

In some embodiments, as shown in FIGS. 3, 4, 6, and 7, a bayonet mount may be used to attach the vibration generator to the patch. In such embodiments, as shown in FIGS. 6 and 7, for example, the vibration generator may include a protrusion 114 that is insertable into a corresponding opening 116 in the patch, such as to align the vibration generator for securement. As will be appreciated, inserting the protrusion into the corresponding opening may not hold the vibration generator to the patch in some embodiments.

Once the protrusion 114 of the vibration generator has been inserted into the corresponding opening 116 of the patch, the user may rotate the vibration generator in a first direction to secure the vibration generator to the patch. As shown in FIG. 2, once rotated, one or more protrusions 118 on the patch (see also FIG. 4) may engage with corresponding openings 120 in the vibration generator (see FIG. 6) to hold the vibration generator on the patch. To remove the vibration generator from the patch, the user may rotate the vibration generator in a second, opposite direction, to disengage the protrusions 118 on the patch from the opening 120 on the vibration generator. Thereafter, the protrusion 114 of the vibration generator may be removed from the corresponding opening 116 in the patch.

Other suitable fasteners may be used to removably attach the vibration generator to the patch. For example, the vibration generator may have clips that engage with corresponding fasteners on the patch. The vibration generator and patch also may have corresponding hook and loop fasteners that engage with one another. In other embodiments, the vibration generator may be fixedly attached or even permanently attached to the patch.

In other embodiments, as shown in FIG. 9, the patch 104 may include a pocket 117 into which the vibration generator is insertable during use. In some embodiments, the pocket may be formed by cutting one or more slits 119 in the patch to form an opening, and thereafter covering at least the opening with a backing layer. For example, in some embodiments a vertical backing strip may overlay only the opening in some embodiments. In other embodiments, the backing layer may be sized to fit the entire patch, with the pocket being formed between the backing layer and the rest of the patch.

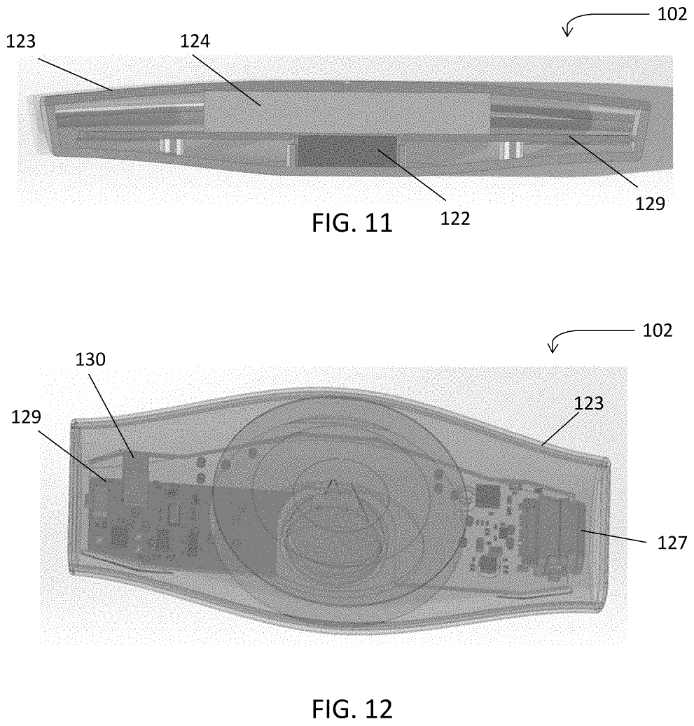

FIGS. 6-8 show various views of a vibration generator 102 according to some embodiments. FIGS. 11 and 12 show various views of a vibration generator 102 according to other embodiments. As shown in these views, the vibration generator may include a housing 123 with a vibration motor 122 for generating the vibration. The vibration generator also may include a battery 124 for powering the vibration motor. In some embodiments, the battery may be rechargeable. In such embodiments, the vibration device may include a charging port 127 (see FIG. 12). The battery also may be replaceable in other embodiments. The vibration generator may include printed circuit board (“PCB”) 129 to which the battery and vibration motor are electrically connected. The charging port also may be connected to the PCB. The vibration generator also may include a control button 126, which may be used to turn the vibration generator on and off and/or to control the vibration generator. For example, the control button may allow the user to change the intensity of the vibration and/or the sequence of vibrations being generated.

In some embodiments, the vibration generator may include a timing function to stop the vibration after a prescribed period of times. In some embodiments, the timing function may include pre-set times for the user to select from. For example, the timing function may allow the user to select vibrations for 1 hour, 2 hours, 3 hours, or more. The timing function also may allow the user to manually set the time for use.

In some embodiments, the vibration generator may be connected to a remote device (e.g., a mobile phone). For example, the vibration generator may be wirelessly connected to the remote device, such as via a wireless, Bluetooth (see Bluetooth connector 130 in FIG. 12), or other suitable connection. In some embodiments, the mobile device may be able to control the operation of the vibration generator. For example, the mobile device may direct the vibration generator to operate at a certain intensity, for a certain period of time, and according to a specific sequence.

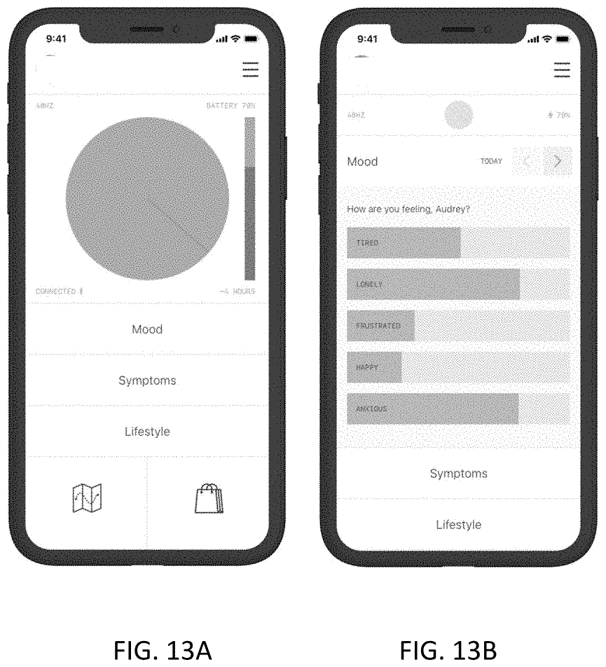

FIGS. 13A and 13B are examples of an interface for a mobile device application (also referred to herein as a mobile application) that a user may use (e.g., on the mobile device) to operate the vibration generator. For example, using the mobile application, the user may select the intensity (e.g., low, medium, high) of the vibration and/or set a vibration pattern of the vibration generator. In such embodiments, the mobile application may then wirelessly communicate the selected parameters to the vibration generator. For example, the mobile application may be connected to the vibration generator via a Bluetooth connection for communicating the selected parameters.

In addition to manually setting the parameters of the vibration generator, the mobile application also may allow the user to track mood, pain symptoms, and/or other lifestyle information. In some embodiments, the application may be arranged to store such information, along with selected vibration parameters. For example, the application may include a module arranged to store the on and off timestamps of the vibration. In such embodiments, the mobile application may create a library of vibration parameters and corresponding pain levels (or other information). In some embodiments, the data may be stored on a remote server (e.g., a cloud). In some embodiments, the vibration generator and mobile application are arranged to sync data to each other when the vibration generator is in proximity of the mobile device.

In some embodiments, the mobile application may use the stored data to predict vibration parameters based on prior use. For example, in response to a user specifying a particular pain level (or other parameter), the mobile application may recommend a particular vibration setting (e.g., medium level vibrations) and/or a particular time (e.g., 1 hour) based on the user's prior use(s). In some embodiments, the mobile application also may be arranged to automatically communicate such recommended vibration parameters to the vibration generator in response to the user indicating a particular pain level (or other parameter).

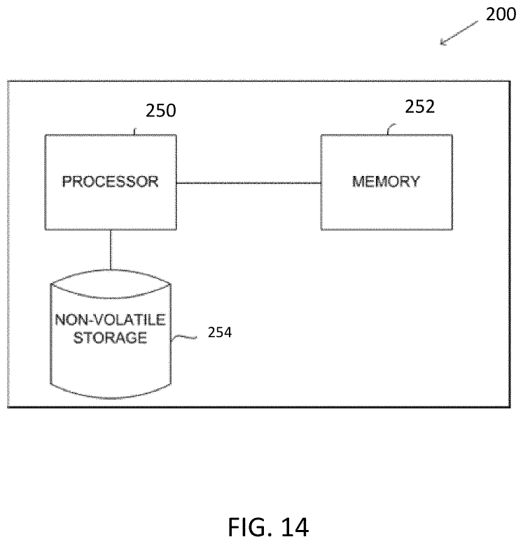

An illustrative implementation of a computer system 200 that may be used in connection with some embodiments of the present invention is shown in FIG. 14. One or more computer systems such as computer system 200 may be used to implement any of the functionality described above. The computer system 200 may include one or more processors 250 (e.g., processing circuits) and one or more computer-readable storage media (i.e., tangible, non-transitory computer-readable media), e.g., volatile storage 252 (e.g., memory) and one or more non-volatile storage media 254, which may be formed of any suitable non-volatile data storage media. The processor(s) 250 may control writing data to and reading data from the volatile storage 252 and/or the non-volatile storage device 254 in any suitable manner, as aspects of the present invention are not limited in this respect. To perform any of the functionality described herein, processor(s) 250 may execute one or more instructions stored in one or more computer-readable storage media (e.g., volatile storage 252), which may serve as tangible, non-transitory computer-readable media storing instructions for execution by the processor 250.

The above-described embodiments of the present invention can be implemented in any of numerous ways. For example, the embodiments may be implemented using hardware, software or a combination thereof. When implemented in software, the software code (e.g., instructions) can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of embodiments of the present invention comprises at least one computer-readable storage medium (i.e., at least one tangible, non-transitory computer-readable medium, e.g., a computer memory, a floppy disk, a compact disk, a magnetic tape, or other tangible, non-transitory computer-readable medium) encoded with a computer program (i.e., a plurality of instructions), which, when executed on one or more processors, performs above-discussed functions of embodiments of the present invention. The computer-readable storage medium can be transportable such that the program stored thereon can be loaded onto any computer resource to implement aspects of the present invention discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-discussed aspects of the present invention.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A pain management device comprising:
a patch having a body with a first side arranged to contact a body of a wearer, a second side opposite the first side, and first and second arms arranged to attach the patch to the body of the wearer, wherein at least a portion of each of the first and second arms is stretchable; and
a vibration generator arranged to be attached to the body of the patch.

2. The apparatus of claim 1, wherein the vibration generator is removably attached to a central region of the second side of the patch, wherein the first and second arms extend outwardly from the central region.

3. The apparatus of claim 1, wherein at least a portion of each of the first and second arms is formed of a stretchable material.

4. The apparatus of claim 1, wherein each of the first and second arms is formed of a stretchable material.

5. The apparatus of claim 1, wherein the patch is formed of a stretchable material.

6. The apparatus of claim 1, wherein at least a portion of the first side of the body is sticky.

7. The apparatus of claim 1, further comprising a plurality of adhesive layers attached to the first side of the body, wherein each of the plurality of adhesive layers is arranged to attach the patch to body of the wearer.

8. The apparatus of claim 1, wherein the vibration generator includes a housing having a vibration motor, a battery, and a circuit board.

9. The apparatus of claim 1, wherein each of the first and second arms includes a stretching zone.

10. The apparatus of claim 9, wherein the stretching zone of each of the first and second arm is formed at a proximal end of the first and second arms.

11. The apparatus of claim 1, wherein the second side includes a mount arranged to attach the vibration generator to the patch.

12. The apparatus of claim 11, wherein the mount includes a bayonet mount.

13. The apparatus of claim 1, wherein at least a portion of the patch is formed of a flexible material.

14. The apparatus of claim 1, wherein the patch has a length of between about 3 inches and about 5.5 inches and a width of between about 2 inches and about 4.5 inches.

15. The apparatus of claim 1, wherein the body includes a pocket arranged to receive the vibration generator.

16. The apparatus of claim 1, wherein the body includes a third arm arranged to attach the patch to the body of the wearer.

17. A kit comprising:
a patch having a body with a first side arranged to contact a body of a wearer, a second side opposite the first side, and first and second arms arranged to attach the patch to the body of the wearer; and
a first vibration generator arranged to be removably attachable to the body of the patch.

18. The kit of claim 17, further comprising a second vibration generator arranged to be removably attachable to the body of the patch.

19. The kit of claim 18, wherein the second side includes a first mount, wherein each of the first and second vibration generators are attachable to the first mount.

20. The kit of claim 18, wherein the second side includes first and second mounts, wherein the first vibration generator is attachable to the first mount and the second vibration generator is attachable to the second mount.

21. The kit of claim 19, wherein the first mount includes a bayonet mount.

22. The kit of claim 18, wherein the body includes a pocket arranged to receive each of the first and second vibration generators.

23. The kit of claim 17, wherein at least a portion of each of the first and second arms is stretchable.

24. The kit of claim 17, wherein at least a portion of the patch is formed of a flexible material.

25. The kit of claim 18, wherein each of the first and second vibration generators includes a housing with a vibration motor, a battery, and a circuit board.

26. A method comprising:
attaching a vibrating pain management device to a body of a wearer, the pain management device including a patch and a vibration generator attachable to the patch, at least a portion of the patch being stretchable; and
operating the vibration generator to provide a vibration to the body of the wearer.

27. The method of claim 26, further comprising attaching the vibration generator to the patch.

28. The method of claim 27, wherein attaching the vibration generator to the patch includes attaching the vibration generator to a mount on the patch or inserting the vibration generator in a pocket of the patch.

29. The method of claim 26, wherein the step of attaching includes attaching first and second arms of the patch to the body of the wearer.

30. The method of claim 29, wherein attaching the first and second arms includes stretching the first and second arms.
